(19) United States
(12) Patent Application Publication
(10) Pub. No.: US 2011/0245517 A1
(43) Pub. Date: Oct. 6, 2011

(54) PROCESS FOR PREPARING TETRAHYDROFURAN

(75) Inventors: Gunther Windecker, Ludwigshafen (DE); Jens Weiguny, Freinsheim (DE); Alexander Weck, Freinsheim (DE); Ellen Dahlhoff, Limburgerhof (DE); Ralf Freyberger, Kuantan (MY)

(73) Assignee: BASF SE, Ludwigshafen (DE)

(21) Appl. No.: 12/740,468

(22) PCT Filed: Nov. 5, 2009
(86) PCT No.: PCT/EP09/64660

§ 371 Date: May 12, 2010

(30) Foreign Application Priority Data
Nov. 17, 2008 (EP) 08169253.5

(51) Int. Cl. C07D 307/08

(52) U.S. Cl. 549/508

(57) ABSTRACT

The present invention relates to a process for preparing tetrahydrofuran by absorption of C₄-dicarboxylic acids and/or derivatives thereof from a crude product mixture into an organic solvent or water as absorption medium, removal of the absorption medium, catalytic hydrogenation of the resulting C₄-dicarboxylic acids and/or derivatives thereof and distillation of the water-comprising crude tetrahydrofuran in at least one distillation column, wherein THF-comprising waste streams from the distillation are catalytically hydrogenated with complete or partial recirculation to the process.

The present invention relates to a process for preparing tetrahydrofuran by absorption of C₄-dicarboxylic acids and/or derivatives thereof from a gaseous crude product mixture into an organic solvent or water as absorption medium, removal of the absorption medium, catalytic hydrogenation of the resulting C₄-dicarboxylic acids and/or derivatives thereof and distillation of the water-comprising crude tetrahydrofuran, wherein the bottom product obtained from the pure distillation of the tetrahydrofuran is catalytically hydrogenated with complete or partial recirculation to the process.

The process of the invention serves to improve the industrial preparation of tetrahydrofuran from maleic anhydride. Maleic anhydride is a valuable starting material, a raw material for polymers or is employed, via hydrogenation of maleic anhydride (MAn) via the intermediate succinic anhydride (SAn), for the preparation of gamma-butyrolactone (GBL), butanediol (BDO) and tetrahydrofuran (THF).

Maleic anhydride can be obtained by partial oxidation of hydrocarbons such as butane or benzene. The desired product is usually absorbed from the maleic anhydride-comprising offgas from the partial oxidation in a solvent.

The gas-phase hydrogenation of MAn forms, with increasing degree of hydrogenation, the products succinic anhydride, GBL, THF, butanol and butane in succession. If THF is isolated as product, all the substances are present in small amounts in addition to the water of hydrogenation formed in the output from the hydrogenation. In addition, further butanol and phthalic acid and its anhydride can be introduced into the hydrogenation from the solvent employed in the prior isolation of MAn.

In addition, lower alcohols such as ethanol and methanol are formed by secondary reactions in the isolation of MAn and in the hydrogenation. The secondary components n-butyraldehyde and butyl methyl ester which are formed in the hydrogenation and are difficult to separate from THF by distillation are particularly critical.

DE-A 37 26 805 and DE-A 10 209 632 disclose distillation processes in which the crude tetrahydrofuran is conveyed through three distillation columns and the pure tetrahydrofuran is isolated via the side offtake of the third column, which serves as pure distillation, or as overhead product therefrom. However, an in-specification THF can only be obtained by these processes if a waste stream still having a high THF content is taken off as bottom product. This bottom product which has hitherto been obtained as waste stream still comprises over 90% by weight of THF as well as up to 0.5% by weight of the abovementioned secondary components. Isolation of the pure THF by removal of the secondary components by distillation therefore results in high THF losses of up to 5% of the total amount.

Proceeding from this prior art, it was an object of the present invention to avoid the high THF losses in the preparation of in-specification THF.

It has surprisingly been found that a process for preparing tetrahydrofuran by absorption of C₄-dicarboxylic acids and/or derivatives thereof from a crude product mixture into an organic solvent or water as absorption medium, removal of the absorption medium, catalytic hydrogenation of the resulting C₄-dicarboxylic acids and/or derivatives thereof and distillation of the water-comprising crude tetrahydrofuran in at least one distillation column, wherein THF-comprising waste streams from the distillation are catalytically hydrogenated with complete or partial recirculation to the process, achieves this object.

The process of the invention makes it possible to avoid the THF losses in the distillation virtually completely by recirculating THF-comprising waste streams from the distillation to the removal of the absorbent or the hydrogenation of the C₄-dicarboxylic acids and/or derivatives thereof, with recirculation to the removal of the absorption medium being preferred. As a result of the recirculation, the major part (>95%) of the THF comprised in these streams is recovered without the specification of the pure product being adversely affected. Surprisingly, the theoretically expected accumulation of the butyraldehyde formed in the hydrogenation and comprised in the THF-comprising waste stream does not occur.

For the purposes of the present patent application, the expression C₄-dicarboxylic acids and derivatives thereof refers to maleic acid and succinic acid, which may optionally have one or more C₁-C₆-alkyl substituents, and also the anhydrides of these optionally alkyl-substituted acids. An example of such an acid is citraconic acid. Preference is given to using the respective anhydrides of a given acid. In particular, the starting material used is maleic anhydride (MAn).

The process of the invention can comprise a preceding step comprising the preparation of an MAn-comprising crude product mixture by partial oxidation of a suitable hydrocarbon. Suitable hydrocarbon streams are benzene, C₄-olefins (e.g. n-butenes, C₄-raffinate streams) or n-butane. Particular preference is given to using n-butane since it represents an inexpensive, economical starting material. Processes for the partial oxidation of n-butane are described, for example, in Ullmann's Encyclopedia of Industrial Chemistry, 6^{th} Edition, Electronic Release, Maleic and Fumaric Acids—Maleic Anhydride.

The reaction output obtained in this way, viz. the crude product mixture, is then taken up in water or preferably in a suitable organic solvent or a mixture thereof as absorption medium, with the organic solvent preferably having a boiling point which is at least 30° C. higher than that of MAn at atmospheric pressure.

The gas stream comprising maleic anhydride from the partial oxidation can be brought into contact with the solvent (absorption medium) in a variety of ways at pressures (absolute) of from 0.8 to 10 bar and temperatures of 50-300° C. in one or more absorption stages: (i) introduction of the gas stream into the solvent (e.g. via gas introduction nozzles or sparging rings), (ii) spraying of the solvent into the gas stream and (iii) countercurrent contact between the upward-flowing gas stream and the downward-flowing solvent in a tray column or packed column. In all three variants, the apparatuses known to those skilled in the art for gas absorption can be used. When choosing the solvent (absorption medium) to be used, care should be taken to ensure, especially in the isolation of MAn, that it does not react with the starting material, viz. the MAn used. Suitable absorption media are: tricresyl phosphate, dibutyl maleate, butyl maleate, high molecular weight waxes, aromatic hydrocarbons having a molecular weight in the range from 150 to 400 and a boiling point above 140° C., for example dibenzylbenzene; alkyl phthalates and dialkyl phthalates having C₁-C₁₈-alkyl groups, for example dimethyl phthalate, diethyl phthalate, dibutyl phthalate, di-n-propyl and diisopropyl phthalate, undecyl phthalate, diundecyl phthalate, methyl phthalate, ethyl phthalate, butyl phthalate, n-propyl or isopropyl phthalate; di-C₁-C₄-alkyl esters of other aromatic and aliphatic dicarboxylic acids, for example dimethyl 2,3-naphthalenedicarboxylate, dimethyl 1,4-cyclohexanedicarboxylate; C₁-C₄-alkyl esters of other aromatic and aliphatic dicarboxylic acids, for example methyl 2,3-naphthalene dicarboxylate, methyl 1,4-cyclohexane dicarboxylate, methyl esters of long-chain fatty acids having, for example, from 14 to 30 carbon atoms, high-boiling ethers, for example dimethyl ethers of polyethylene glycol, for example tetraethylene glycol dimethyl ether.

The use of phthalates is preferred.

The solution obtained after the treatment with the absorption medium generally has an MAn content of from about 5 to 400 gram per liter.

The offgas stream remaining after the treatment with the absorption medium comprises mainly the by-products of the preceding partial oxidation, e.g. carbon monoxide, carbon dioxide, unreacted butanes, acetic acid and acrylic acid, in addition to water. The offgas stream is virtually free of MAn.

The dissolved MAn is subsequently stripped or separated off from the absorption medium by distillation. The removal of the absorption medium is preferably carried out by stripping with hydrogen at a pressure which is not more than 10% above the pressure of a subsequent hydrogenation of the MAn to THF, BDO or GBL, preferably at from 100 to 250° C. and pressures (absolute) of from 0.8 to 30 bar. A temperature profile resulting from the boiling points of MAn at the top and the virtually MAn-free absorption medium at the bottom of the column at the respective column pressure and the dilution with carrier gas (hydrogen) set is observed in the stripping column. To prevent losses of solvent, rectification internals can be present above the inlet for the crude MAn stream. The hydrogen is preferably circulated between hydrogenation and stripping column (circulating gas).

The hydrogen/maleic anhydride stream obtained in this way is then fed to the hydrogenation zone. The catalytic hydrogenation of the C₄-dicarboxylic acids and/or derivatives thereof is preferably carried out by the process which is comprehensively described in WO 02/48 128, which is hereby expressly incorporated by reference. The hydrogenation is accordingly preferably carried out in the gas phase using a catalyst comprising <80% by weight, preferably <70% by weight, in particular from 10 to 65% by weight, of CuO and >20% by weight, preferably >30% by weight, in particular from 35 to 90% by weight, of an oxidic support having acid sites, with the process being carried out at a hot spot temperature of from 240 to 310° C., preferably from 240 to 280° C., and WHSVs over the catalyst of from 0.01 to 1.0, preferably from 0.02 to 1, in particular from 0.05 to 0.5, kg of starting material/l of catalyst·hour. The catalyst according to WO 02/48 128 comprises copper oxide as catalytically active main constituent. This is applied to an oxidic support which has to have a suitable number of acid sites. The required amount of oxidic support depends on the number of acidic sites present therein. A suitable support material having a sufficient number of acidic sites is aluminum oxide, whose use is preferred according to one embodiment of the present invention. In another embodiment of the present invention, preference is given to using a combination of aluminum oxide with zinc oxide in a weight ratio of from 20:1 to 1:20, preferably from 5:1 to 1:5, as acidic support material. In the case of materials which have a large number of such acidic sites, the lower limit to the amount of support comprising such a material is 20% by weight. The amount of copper oxide is <80% by weight. Preferred catalyst compositions comprise <70% by weight of copper oxide and >30% by weight of support, and particularly preferred catalysts comprise from 10 to 65% by weight of copper oxide and from 35 to 90% by weight of support. The catalysts used according to the invention, which are chromium-free, can optionally comprise one or more further metals or compounds thereof, preferably an oxide, from groups 1 to 14 (IA to VIIIA and IB to IVB according to the old IUPAC nomenclature) of the Periodic Table of the Elements. If such a further oxide is used, preference is given to using TiO₂, ZrO₂, SiO₂ and/or MgO.

The catalysts used can additionally comprise an auxiliary in an amount of from 0 to 10% by weight. For the purposes of the present invention, auxiliaries are organic and inorganic materials which contribute to improved processing during catalyst production and/or to an increase in the mechanical strength of the shaped catalyst bodies. Such auxiliaries are known to those skilled in the art; examples comprise graphite, stearic acid, silica gel and copper powder.

The catalysts can be produced by methods known to those skilled in the art, as described in WO 02/48 128.

An important parameter in the hydrogenation is adherence to a suitable reaction temperature. This is achieved, firstly, by a sufficiently high inlet temperature of the starting materials. This temperature is from >220 to 300° C., preferably from 235 to 270° C. To obtain an acceptable or high THF selectivity and yield, the reaction has to be carried out so that a suitably high reaction temperature prevails over the catalyst bed over which the actual reaction takes place. This temperature known as the hot spot temperature is established after entry of the starting materials into the reactor and is in the range from 240 to 310° C., preferably from 240 to 280° C. The process is carried out so that the inlet temperature and the outlet temperature of the reaction gases are below this hot spot temperature. The hot spot temperature is advantageously located in the 1st half of the reactor, in particular when the reactor is a shell-and-tube reactor. The hot spot temperature is preferably from 5 to 15° C. above, in particular from 10 to 15° C. above, the inlet temperature. If the hydrogenation is carried out below the minimum temperatures of the inlet temperature or hot spot temperature, then when MAn is used as starting material, the amount of GBL increases while the amount of THF decreases. Furthermore, deactivation of the catalyst due to coating with succinic acid, fumaric acid and/or SAn is observed at such a temperature during the course of the hydrogenation. If, on the other hand, MAn as starting material is hydrogenated above the maximum temperatures of the inlet temperature or hot spot temperature, the THF yield and selectivity drop to unsatisfactory values. In this case, increased formation of n-butanol and n-butane, i.e. the products of overhydrogenation, is observed. The WHSV over the catalyst in the hydrogenation according to the invention is in the range from 0.01 to 1.0 kg of starting material/l of catalyst·hour. From an economic point of view, a low hydrogen/starting material ratio is desirable. The lower limit is 5, but higher hydrogen/starting material molar ratios of from 20 to 400 are generally employed. The use of the above-described catalysts according to the invention and adherence to the above-described temperature values allows the use of advantageous, low hydrogen/starting material ratios which are preferably in the range from 20 to 200, preferably from 40 to 150. The most advantageous range is from 50 to 100.

To set the hydrogen/starting material molar ratios used according to the invention, part, advantageously the major part, of the hydrogen is circulated. For this purpose, the circulating gas compressors known to those skilled in the art are generally used. The amount of hydrogen which is chemically consumed by the hydrogenation is replaced. In a preferred embodiment, part of the circulating gas is discharged in order to remove inert compounds, for example n-butane. The circulated hydrogen can then also be utilized, if appropriate after preheating, for vaporizing the feed stream.

The volume flow of the reaction gases, generally expressed as GHSV (gas hourly space velocity), is also an important parameter in the process of the invention. The GHSVs in the process of the invention are in the range from 100 to 10 000 standard n³/m³h, preferably from 1000 to 3000 standard n³/m³h, in particular from 1100 to 2500 standard n³/m³h. The pressure at which the hydrogenation according to the invention is carried out is in the range from 1 to 30 bar, preferably from 2 to 9 bar, in particular from 3 to 7 bar.

The hydrogenation step according to the invention is preferably carried out in one or more separate reactors. Preference is given to using at least one tube reactor, for example at least one shaft reactor and/or at least one shell-and-tube reactor, for the hydrogenation, with an individual reactor being able to be operated in the upflow mode or the downflow mode. When two or more reactors are used, at least one can be operated in the upflow mode and at least one can be operated in the downflow mode.

The gas stream leaving the reactor is cooled to from 10 to 60° C. Here, the reaction products are condensed out and passed to a separator. The uncondensed gas stream is taken off from the separator and fed to the circulating gas compressor. A small amount of circulating gas is discharged. The condensed-out hydrogenation product, viz. the crude water-comprising THF, is continuously taken off from the system and passed to the work-up. The crude water-comprising THF which has been obtained by gas-phase hydrogenation of MAn generally comprises 61% by weight of THF, 4% by weight of n-butanol (n-BuOH), 0.7% by weight of methanol (MeOH), 0.5% by weight of ethanol (EtOH), 1% by weight of propanol (ProOH), 400 ppm of gamma-butyrolactone (GBL), 120 ppm of butyraldehyde (BA), 100 ppm of butyl methyl ether (BME), further O-functionalized CH compounds in concentrations of <200 ppm and also water. The crude water-comprising THF is then purified by means of distillation in at least one distillation column.

The THF-comprising waste streams from the distillation obtained during the work-up of the crude water-comprising THF by distillation can, according to the process of the invention, be recirculated in a proportion of from 0.1 to 99%, preferably 75%, to the above-described preparation of the THF, in particular to the removal of the absorption medium or the hydrogenation of the C₄-dicarboxylic acids and/or derivatives thereof. These THF-comprising waste streams from the distillation generally comprise up to 99% by weight of THF, up to 2% by weight of butanol, ethanol, propanol, GBL and 3-methyl-THF and up to 5% of n-butyraldehyde and butyl methyl ester.

The crude water-comprising THF is preferably purified by means of a distillation using three columns, as is described, for example, in DE-A 37 26 805 and DE-A 102 09 632. As THF-comprising waste streams from the distillation of the crude water-comprising THF, preference is given to the bottom products. In a distillation using three distillation columns, the bottom products from the first and third columns are possibilities, with the bottom product from the third column being particularly preferred.

Particular preference is therefore given to using the bottom product from the third column of the distillation process as described in DE-A 37 26 805 or DE-A 102 09 632, which in each case serves for the pure distillation of the THF, in the process of the invention. This bottom product generally comprises up to 99% by weight of THF, up to 0.5% by weight of butanol, ethanol, propanol, GBL and water and up to 2% of n-butyraldehyde and butyl methyl ester and also traces of methyl-THF.

This bottom product from the third column is particularly preferably obtained as described in DE-A 102 09 632 by passing the crude water-comprising tetrahydrofuran through three distillation columns, taking off water from the bottom of the first column, recirculating water-comprising tetrahydrofuran from the top of the second column to the first column, feeding a side offtake stream from the first column to the second column, recirculating the bottom product of the third column to the first column, taking off a distillate at the top of the first column, with a side offtake stream from the second column being fed to the third column and the pure tetrahydrofuran being obtained as overhead product from the third column and the bottom product also being obtained. This process for purification by distillation is comprehensively described in DE-A 102 09 632, which is hereby expressly incorporated by reference.

The bottom product from the third column of the distillation of the water-comprising crude THF can be recirculated as THF-comprising waste stream to the removal of the absorption medium or the hydrogenation of the C₄-dicarboxylic acids and/or derivatives thereof. The bottom product is preferably recirculated to the above-described removal of the absorption medium prior to the hydrogenation of the C₄-dicarboxylic acids and/or derivatives thereof and fed together with the C₄-dicarboxylic acids and/or derivatives thereof obtained after removal of the absorption medium to the catalytic hydrogenation to form THF. This manner of recirculation is advantageous since a dedicated vaporizer unit for the bottom product of the third column and a separate hydrogenation are saved.

However, it is also possible to recirculate the bottom product from the third column of the distillation of the water-comprising crude THF directly to the catalytic hydrogenation of the C₄-dicarboxylic acids and/or derivatives thereof in the hydrogenation zone. Here, the bottom product is firstly vaporized and then preferably mixed with the hydrogen/maleic anhydride steam from the removal of absorption medium upstream of or in the hydrogenation zone.

Apart from the hydrogenation zone for the hydrogenation of the C₄-dicarboxylic acids and/or derivatives thereof, the bottom product from the third column can also be catalytically hydrogenated in a separate hydrogenation zone which can comprise one or more separate hydrogenation reactors and then be recirculated to the distillation. In a particularly preferred embodiment, the separate hydrogenation reactor for the hydrogenation stage of the process of the invention is supplied with offgas hydrogen from the hydrogenation of MAn to THF. The recirculation is, in the case of the preferred distillation arrangement having three distillation columns, preferably carried out to the first column, but recirculation to the second column is likewise possible.

Recirculation to the removal of the absorption medium before the hydrogenation of the C₄-dicarboxylic acids and/or derivatives thereof is preferred.

The hydrogenation of the bottom product in the separate hydrogenation zone is carried out in the liquid phase over heterogeneous catalysts which can be present as a fixed bed or in suspension, with fixed-bed catalysts being preferred.

The catalysts which can be used preferably comprise at least one metal of groups 7, 8, 9, 10 or 11 of the Periodic Table of the Elements or a compound thereof, for example an oxide. More preferably, the catalysts which can be used according to the invention comprise at least one element selected from the group consisting of Re, Fe, Ru, Co, Rh, Ir, Ni, Pd, Pt, Cu and Au. The catalysts which can be used according to the invention particularly preferably comprise at least one element selected from the group consisting of Ni, Pd, Pt, Ru and Cu. Very particular preference is given to the catalysts which can be used according to the invention comprising Pd, Pt, Ru or Ni.

A suitable catalyst is, in particular, at least one heterogeneous catalyst, with at least one of the abovementioned metals (active metals) being able to be used as metal, as Raney catalyst and/or applied to a customary support. If two or more active metals are used, they can be present separately or as an alloy. It is possible to use at least one metal as such and at least one other metal as Raney catalyst or at least one metal as such and at least one other metal applied to at least one support or at least one metal as Raney catalyst and at least one other metal applied to at least one support or at least one metal as such and at least one other metal as Raney catalyst and at least one other metal applied to at least one support.

The catalysts used can also be, for example, precipitated catalysts. Such catalysts can be produced by precipitating their catalytically active components from their salt solutions, in particular from the solutions of their nitrates and/or acetates, for example by addition of solutions of alkali metal and/or alkaline earth metal hydroxide and/or carbonates, for example as sparingly soluble hydroxides, hydrated oxides, basic salts or carbonates, subsequently drying the precipitates obtained and then converting these by calcination at generally from 300 to 700° C., in particular from 400 to 600° C., into the corresponding oxides, mixed oxides and/or mixed-valency oxides which are reduced by treatment with hydrogen or with hydrogen-comprising gases in the range of generally from 50 to 700° C., in particular from 100 to 400° C., to the corresponding metals and/or oxidic compounds in a lower oxidation state and converted into the actual catalytically active form. The reduction is generally continued until no more water is formed. In the production of precipitated catalysts comprising a support material, the precipitation of the catalytically active components can be carried out in the presence of the support material concerned. The catalytically active components can advantageously be coprecipitated with the support material from the salt solutions concerned.

Preference is given to using hydrogenation catalysts which comprise the metals or metal compounds which catalyze the hydrogenation on a support material.

Apart from the abovementioned precipitated catalysts which comprise a support material in addition to the catalytically active components, support materials in which the catalytically hydrogenatively active component has been applied to a support material, for example by impregnation, are generally suitable for the process of the invention.

The manner in which the catalytically active metal is applied to the support is generally not critical and can be effected in a variety of ways. The catalytically active metals can be applied to these support materials by, for example, impregnation with solutions or suspensions of the salts or oxides of the elements concerned, drying and subsequent reduction of the metal compounds to the respective metals or compounds of a lower oxidation state by means of a reducing agent, for example by means of hydrogen or complex hydrides. Another possible way of applying the catalytically active metals to these supports is to impregnate the supports with solutions of salts which can readily be decomposed thermally, for example nitrates, or complexes which can easily be decomposed thermally, for example carbonyl or hydrido complexes, of the catalytically active metals and to heat the support which has been impregnated in this way to temperatures in the range from 300 to 600° C. to bring about a thermal decomposition of the adsorbed metal compounds. This thermal decomposition is preferably carried out under a protective gas atmosphere. Suitable protective gases are, for example, nitrogen, carbon dioxide, hydrogen or the noble gases. Furthermore, the catalytically active metals can be deposited on the catalyst support by vapor deposition or by flame spraying. The content of the catalytically active metals in these supported catalysts is in principle not critical to the success of the process of the invention. In general, higher contents of catalytically active metals in these supported catalysts lead to higher space-time yields than lower contents. In general, use is made of supported catalysts whose content of catalytically active metals is in the range from 0.01 to 90% by weight, preferably in the range from 0.1 to 40% by weight, based on the total weight of the catalyst. Since these content figures are based on the total catalyst including support material but the various support materials have very different specific gravities and specific surface areas, it is also conceivable for the amounts employed to be below or above these figures without this having an adverse effect on the result of the process of the invention. It is of course also possible for a plurality of the catalytically active metals to have been applied to the respective support material. Furthermore, the catalytically active metals can be applied to the support by, for example, the process of DE-A 25 19 817, EP-A 1 477 219 or EP-A 0 285 420. In the catalysts according to the abovementioned documents, the catalytically active metals are present in the form of alloys which are produced by thermal treatment and/or reduction of the support material obtained, for example, by impregnation with a salt or complex of the abovementioned metals.

Owing to the toxicity of chromium-comprising catalysts, preference is given to using chromium-free catalysts. Of course, appropriate industrial chromium-comprising catalysts known to those skilled in the art are also possible for use in the process of the invention, but these do not give the desired advantages, in particular in terms of environmental protection and occupational hygiene.

The activation of both the precipitated catalysts and the supported catalysts can also be carried out in situ at the beginning of the reaction by means of the hydrogen present. These catalysts are preferably activated separately before use.

As support materials both for precipitated catalysts and for supported catalysts, it is possible to use the oxides of aluminum and titanium, zirconium dioxide, silicon dioxide, clay minerals such as montmorillonites, bentonites, silicates such as magnesium or aluminum silicates, zeolites such as the structure types ZSM-5 or ZSM-10 or activated carbon. Preferred support materials are aluminum oxides, titanium dioxides, silicon dioxide, zirconium dioxide and activated carbon. It is of course also possible for mixtures of various support materials to serve as supports for catalysts used in the process of the invention. Metallic supports onto which the hydrogenation-active metal has been deposited, for example Cu onto which, for example, Pd, Pt or Ru has been deposited from the corresponding metal salts dissolved in water, are also suitable.

Particularly preferred catalysts according to the invention are supported catalysts comprising Ni, Pt and/or Pd, with particularly preferred supports being activated carbon, aluminum oxide, titanium dioxide and/or silicon dioxide or mixtures thereof.

A heterogeneous catalyst which can be used according to the invention can be used as suspended catalyst and/or as fixed-bed catalyst in the process of the invention.

The process of the invention can be carried out batchwise, semicontinuously or continuously. It is preferably carried out continuously.

The process of the invention is illustrated by the following examples.

### EXAMPLES

### Example 1

### 1a) Experimental Plant

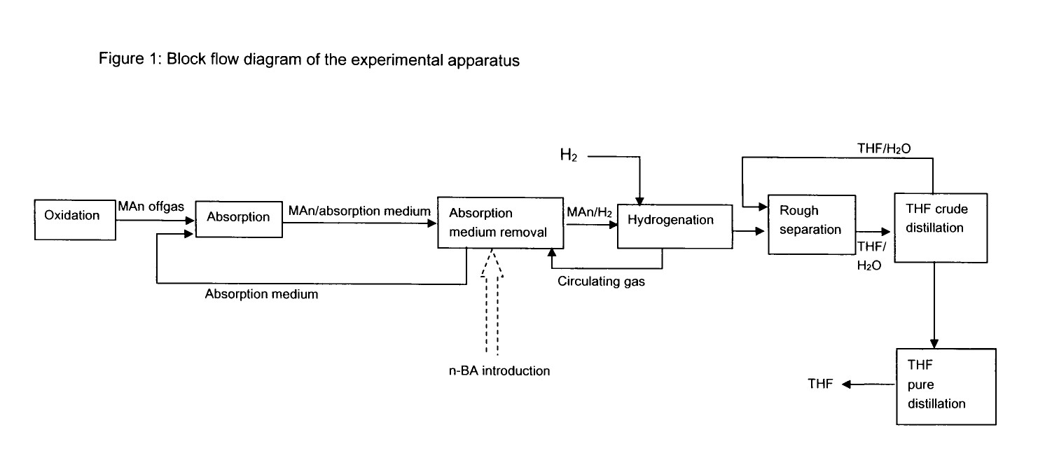

The experimental plant comprises an oxidation reactor, an absorption column for separating the MAn from the offgas from the oxidation reactor by means of dibutyl phthalate (DBP) as solvent, a column for stripping the maleic anhydride (MAn) from the solvent by means of hydrogen, the hydrogenation reactor in which MAn is hydrogenated to THF and the secondary components and an arrangement of three columns corresponding to DE 10209632 for the pure distillation of the THF. The experimental plant is shown schematically in FIG. 1. A stream composed of 99.8% by weight of THF, 0.1% by weight of butanol and 0.1% by weight of n-BA (“n-BA introduction”) is fed into the absorption column. The composition of this stream corresponds to the actual composition of a bottom product from the third column of the experimental plant and simulates the recirculation of this bottom product as THF-comprising waste stream.

### 1b) Experimental Procedure

The oxidation reactor was operated at a pressure of 2.9 bar and a temperature of 403° C. using 41.2 kg/h of air and 1.4% of butane at a conversion of about 83%. The MAn produced in the oxidation was absorbed in dibutyl phthalate in the absorption column. The resulting solution comprising 9.5% by weight of MAn in dibutyl phthalate was mixed with a stream of 60 g/h comprising 99.8% of THF, 0.1% of butanol and 0.1% of n-BA (n-butyraldehyde) so that a n-BA concentration of about 0.3% by weight of n-BA was established in the feed to the stripper.

This gave a concentration of 0.0125% by weight of n-BA in the output from the hydrogenation and a concentration of 1.7-2% by weight in the bottoms from the third column at an offtake rate which corresponded to a simulated THF-comprising waste stream (bottom product from the third column) of 60 g/h. The maximum concentration of n-BA in the hydrogenation output is thus less than the simulated concentration in the THF-comprising waste stream. When the recirculation is carried out in this way, n-BA is therefore converted into unproblematical compounds.

The yield of THF in the distillation was 97.5%.

### Comparative Example 2

### 2a) Experimental Apparatus

The experimental apparatus in this experiment corresponds to the experimental apparatus described under 1a), but no stream composed of 99.8% by weight of THF, 0.1% by weight of butanol and 0.1% by weight of n-BA (“n-BA introduction”) is fed into the absorption column.

### 2b) Experimental Procedure

The oxidation, absorption and stripping were carried out using the same parameters as in experiment 1. However, no n-BA introduction was carried out here. An n-BA concentration of 0.0182% of n-BA was obtained in the hydrogenation output and 1.8% of n-BA were present in the bottoms from the third column. This concentration corresponds to the steady-state concentration with recirculation as described in example 1. n-BA is formed in equilibrium with its starting materials in the hydrogenation and the steady-state concentration in the hydrogenation output is independent of the n-BA concentration in the hydrogenation input in the relevant concentration range.

The yield of THF in the distillation was 99.5%.

Comparison of the experiment according to the invention and the comparative example shows that no accumulation of the by-products occurs as a result of the recirculation. The THF yield could be increased by 2% by recovery of THF from waste streams.

1. A process for preparing tetrahydrofuran, comprising absorbing a C₄-dicarboxylic acid and/or derivative thereof from a crude product mixture into an organic solvent or water as absorption medium, removing the absorption medium, catalytically hydrogenating the resulting C₄-dicarboxylic acid and/or derivative thereof, and distilling the water-comprising crude tetrahydrofuran in at least one distillation column, wherein THF-comprising waste streams from the distillation are catalytically hydrogenated with complete or partial recirculation to the process.

2. The process according to claim 1, wherein the distillation is carried out in three columns and the bottom product from the tetrahydrofuran pure distillation (third column) is the THF-comprising waste stream.

3. The process according to claim 2, wherein the crude tetrahydrofuran is passed through three distillation columns, water is taken off from the bottom of the first column, water-comprising tetrahydrofuran from the top of the second column is recirculated to the first column, a side offtake stream from the first column is fed to the second column, the bottom product from the third column is recirculated to the first column, a distillate is taken off at the top of the first column, a side offtake stream from the second column is fed to the third column and the pure tetrahydrofuran is obtained as overhead product from the third column.

4. The process according to claim 2, wherein the bottom product is recirculated to the absorption medium removal before the hydrogenation of the C₄-dicarboxylic acids and/or derivatives thereof.

5. The process according to claim 2, wherein the bottom product is recirculated to the catalytic hydrogenation of the C₄-dicarboxylic acids and/or derivatives thereof.

6. The process according to claim 2, wherein the bottom product is catalytically hydrogenated in a separate hydrogenation reactor and the hydrogenation output is recirculated to the distillation.

7. The process according to claim 1, wherein the hydrogenation is carried out in the gas phase over a catalyst comprising <80% by weight, of CuO and >20% by weight, of an oxidic support having acid sites, with the process being carried out at a hot spot temperature of from 240 to 310° C., and WHSVs over the catalyst of from 0.01 to 1.0, kg of starting material/l of catalyst·hour.

8. The process according to claim 7, wherein the oxidic support is aluminum oxide or a combination of aluminum oxide with zinc oxide in a weight ratio of from 20:1 to 1:20.

9. The process according to claim 1, wherein the crude product mixture comprises maleic anhydride prepared by oxidation of benzene, C₄-olefins or n-butane.

10. The process according to claim 9, wherein the maleic anhydride is separated from the absorption medium by distillation or by stripping with hydrogen.

11. The process according to claim 1, wherein the absorption medium is selected from the group consisting of tricresyl phosphate, dibutyl maleate, a high molecular weight wax, an aromatic hydrocarbon having a molecular weight in the range from 150 to 400 and a boiling point above 140° C., a di-C₁-C₄-alkyl ester of aromatic and aliphatic dicarboxylic acid, a methyl ester of a long-chain fatty acid having from 14 to 30 carbon atoms, a high-boiling ester, and an alkyl phthalate and dialkyl phthalate having C₁-C₁₈-alkyl groups.

12. The process according to claim 1, wherein the maleic anhydride is driven off from the absorption medium under reduced pressure or at pressures which correspond to the pressure of the hydrogenation or not more than 10% above this pressure.

13. The process according to claim 1, wherein the process is carried out batchwise, semicontinuously or continuously.

14. The processing according to claim 11, wherein the alkyl phthalate or dialkyl phthalate having C₁-C₁₈-alkyl groups is selected from the group of dimethyl phthalate, diethyl phthalate, dibutyl phthalate, di-n-propyl and diisopropyl phthalate, undecyl phthalate, diundecyl phthalate, methyl phthalate, ethyl phthalate, butyl phthalate, n-propyl and isopropyl phthalate.
